(19) United States
(12) Patent Application Publication
(10) Pub. No.: US 2025/0221666 A1
(43) Pub. Date: Jul. 10, 2025

(54) WEARABLE DEVICE WITH ENERGY HARVESTING MODULE

(71) Applicant: Oura Health Oy, Oulu (FI)

(72) Inventors: Tero Juhani Vallius, Kontio (FI); Jussi Petteri Järvelä, Kempele (FI); Jukka Tapani Mäkinen, Oulu (FI); Arto Michael Drees, Oulu (FI); Jaakko Tapio Vartiainen, Oulu (FI); Antti Kalevi Lämsä, Oulu (FI); Tiina Seebeck, Oulu (FI); Mari Määttälä, Oulu (FI); Sami Sakari Ihme, Oulu (FI); Jouni Juhani Huopana, Oulu (FI); Olli Petteri Heikkinen, Oulu (FI); Mikko Alikärri, Helsinki (FI)

(21) Appl. No.: 19/006,814

(22) Filed: Dec. 31, 2024

Related U.S. Application Data
(60) Provisional application No. 63/617,606, filed on Jan. 4, 2024.

(51) Int. Cl. A61B 5/00; H01L 25/16; H10N 10/13

(52) U.S. Cl. CPC A61B 5/6826; H01L 25/167; H10N 10/13; A61B 2560/0214; A61B 2560/0443; A61B 2562/18

(57) ABSTRACT

Methods, systems, and devices for energy harvesting within a wearable device are described. The wearable device may include an inner ring-shaped housing, one or more sensors, and an outer ring-shaped housing. The outer ring-shaped housing may include at least one thermally isolated portion that extends at least partially around the wearable ring device and is isolated from a remaining portion of the outer ring-shaped housing. The device may further include one or more thermo-electric-generator (TEG) components disposed between the inner ring-shaped housing and the at least one thermally isolated portion of the outer ring-shaped housing. The one or more TEG components are configured to generate an electric current to power the one or more sensors, recharge a battery of the wearable ring device, or both, based on a temperature difference between the inner ring-shaped housing and the at least one thermally isolated portion of the outer ring-shaped housing.

### CROSS-REFERENCE

The present application for patent claims the benefit of U.S. Provisional Patent Application No. 63/617,606 by VALLIUS et al., entitled “WEARABLE DEVICE WITH ENERGY HARVESTING MODULE,” filed Jan. 4, 2024, assigned to the assignee hereof, and expressly incorporated by reference herein.

### FIELD OF TECHNOLOGY

The following relates to wearable devices and data processing, including wearable devices with energy harvesting modules.

### BACKGROUND

Some wearable devices may be configured to collect data from users to help the users understand more about their overall physiological health and well-being. However, the battery life of the wearable device may affect the overall performance and user experience of the wearable device.

### BRIEF DESCRIPTION OF THE DRAWINGS

FIG. 1 illustrates an example of a system that supports wearable devices with energy harvesting modules in accordance with aspects of the present disclosure.

FIG. 2 illustrates an example of a system that supports wearable devices with energy harvesting modules in accordance with aspects of the present disclosure.

FIG. 3 shows an example of a system that supports a wearable device with energy harvesting modules in accordance with aspects of the present disclosure.

FIG. 4 shows an example of a wearable ring device with energy harvesting modules in accordance with aspects of the present disclosure.

FIG. 5 shows a block diagram of an apparatus that supports a wearable device with energy harvesting modules in accordance with aspects of the present disclosure.

FIG. 6 shows a block diagram of a wearable device manager that supports a wearable device with energy harvesting modules in accordance with aspects of the present disclosure.

FIG. 7 shows a diagram of a system including a device that supports wearable devices with energy harvesting modules in accordance with aspects of the present disclosure.

FIG. 8 shows a flowchart illustrating methods that support wearable devices with energy harvesting modules in accordance with aspects of the present disclosure.

### DETAILED DESCRIPTION

Wearable devices are typically battery-powered, and may be recharged via external charging devices. However, wearable devices are only able to collect physiological data while the devices are being worn, and typically have to be taken off and placed on a dedicated charger in order to recharge the devices. As such, conventional wearable devices are unable to collect physiological data while the wearable devices are charging, resulting in gaps in the collected physiological data. These gaps in collected data may prevent the user from gaining a full picture of their overall health, and may prevent a wearable device from detecting important physiological events for a user that occur when the wearable device is charging.

Accordingly, techniques described herein may support a wearable device with an energy harvesting module. Specifically, energy harvesting techniques may be utilized to capture and store energy from external sources in order to recharge and power the wearable device. Such energy harvesting techniques may be used to increase the battery life of the wearable device, reduce the frequency of charging, and therefore reduce interruptions to physiological data collection. For example, the wearable device may implement at least one thermo-electric-generator (TEG) component between the inner and outer thermally-conductive shells of the ring. The TEG component may generate a voltage across a thermocouple based on a temperature difference between the inner and outer shells of the wearable device, where the voltage may be used to generate an electrical current to power the wearable device and/or charge the battery. Specifically, the inner shell may be in direct contact with the warm finger, and the outer shell may be exposed to the colder ambient air, which creates a natural temperature difference over the wearable device and may be used for charging the wearable device. In other cases, the outer shell may be in contact with a hot surface, thereby creating a natural temperature difference across the wearable device relative to the colder skin temperature.

To maximize the temperature difference between the inner and outer shells of the wearable device, and therefore increase the output and/or efficiency of the TEG components, the outer thermally-conductive shell may be divided into two sections. A first section/portion of the outer shell may be in direct contact with adjacent fingers, and a second section/portion of the outer shell may be exposed to the ambient air and thermally isolated from the first section. In such cases, the TEG components may be coupled with the inner shell and the thermally isolated portion of the outer shell. By segmenting and thermally isolating the portion of the outer shell that is in contact with the TEG components, the temperature difference between the inner shell and the second section of the outer shell may be maximized by reducing or eliminating heat from adjacent fingers from increasing the temperature of the second section of the outer shell, thereby increasing the efficiency and electrical output of the TEG components.

For the purposes of the present disclosure, the term “curved,” “circumferential,” and like terms, may be used interchangeably to refer to any surface or shape that exhibits a curved profile or contour. As such, the terms “curved” and “circumferential,” may be used to refer to surfaces/shapes that are circular, elliptical, etc., unless noted otherwise herein. Similarly, the term “circumference” may be used to refer to the shape of a wearable ring device that wraps radially around a user's finger (e.g., 360° perimeter or shape), and is not to be interpreted as referring solely to a perfectly circular shape. That is, the term “circumference” may be used to refer to any radial span that extends radially (e.g., 360°) around the ring. For example, a wearable ring device may be said to have a “circumference” that wraps radially around the user's finger even in cases where the ring itself is not a perfect circle. That is, warble ring devices with an elliptical shape, flat portions, etc. may still be said to exhibit a “circumference” in that the wearable ring devices exhibit a shape/perimeter that wraps around a user's finger.

Aspects of the disclosure are initially described in the context of systems supporting physiological data collection from users via wearable devices. Aspects of the disclosure are further illustrated in cross-sectional views of a wearable ring device. Aspects of the disclosure are further illustrated by and described with reference to apparatus diagrams, system diagrams, and flowcharts that relate to wearable devices with energy harvesting modules.

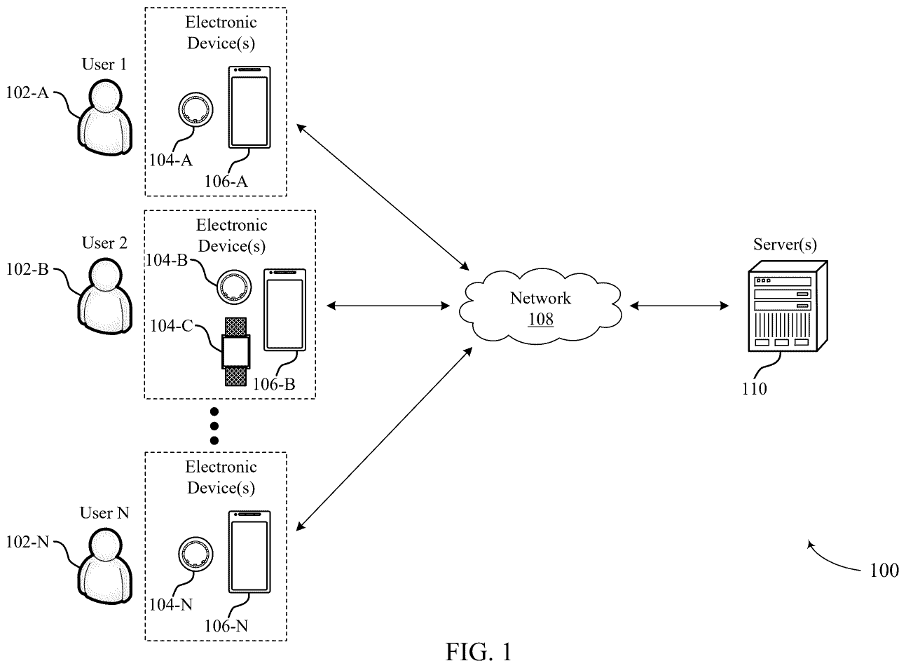

FIG. 1 illustrates an example of a system 100 that supports wearable devices with energy harvesting modules in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may include a ring 104 that exhibits the energy-harvesting features described herein. For example, each ring 104 may include an inner cover and an outer cover. In such cases, each ring 104 may include an inner ring-shaped housing (e.g., the inner cover/shell) defining an inner curved surface (e.g., inner circumferential surface) of the ring 104 and including one or more apertures. The ring 104 may also include an outer ring-shaped housing (e.g., the outer cover/shell) that at least partially surrounds the inner ring-shaped housing and defines an outer curved surface (e.g., outer circumferential surface) of the ring 104. In some cases, electrical components (e.g., PCB, optical sensors, etc.) may be secured or otherwise attached to the inner cover/shell of the ring and configured to acquire physiological data from a user through the apertures.

In some cases, the outer cover/shell may include at least one thermally isolated portion that extends at least partially around the ring 104 and that is thermally isolated from a remaining portion of the outer cover/shell. The ring 104 may further include one or more TEG components at least partially disposed between the inner cover/shell and the at least one thermally isolated portion of the outer cover/shell. The TEG components may be an example of an energy harvesting module that is configured to generate an electric current to power the electrical components, recharge a battery of the ring 104, or both, based on a temperature difference between the inner cover/shell and the at least one thermally isolated portion of the outer cover/shell.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

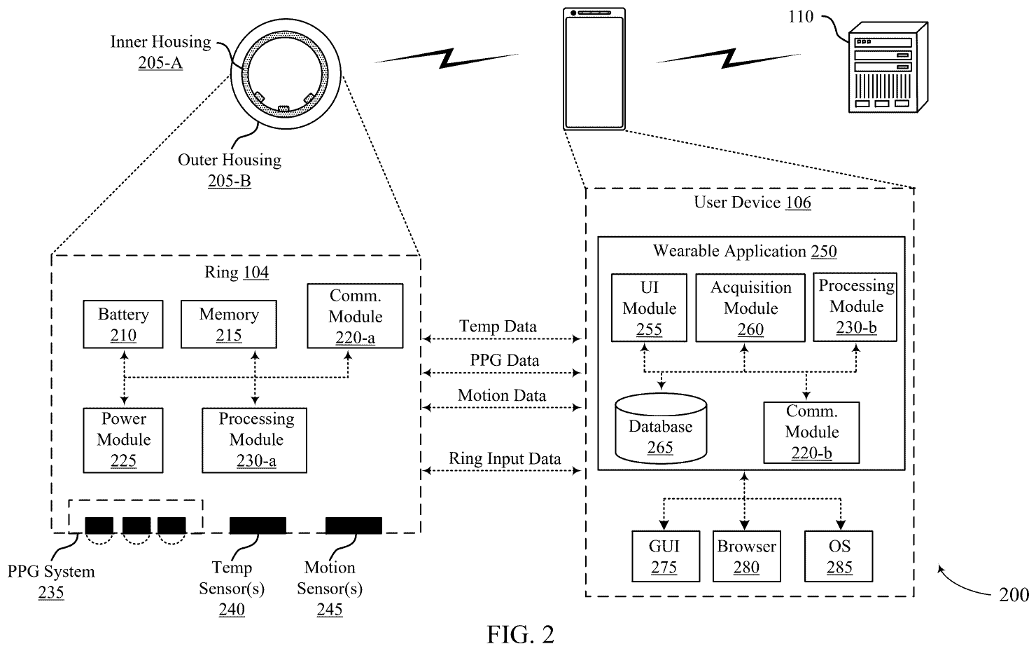

FIG. 2 illustrates an example of a system 200 that supports wearable devices with energy harvesting modules in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some cases, the ring 104 may include one or more TEG components that are at least partially disposed between the inner cover (e.g., inner shell) and an thermally isolated portion of the outer cover (e.g., thermally isolated portion of the outer shell). The TEG components may be configured to generate an electric current to power the one or more sensors (e.g., the memory 215, the communication module 220-a, the power module 225, the processing module 230-a, the PPG system 235, the temperature sensor(s) 240, the motion sensor(s) 245, or any combination thereof), recharge the battery 210, or both, based on a temperature difference between the inner cover and the at least one thermally isolated portion of the outer cover. The TEG components may be an example of an energy harvesting module.

To maximize the temperature difference between the inner cover and the outer cover, and therefore increase the output and/or efficiency of the TEG components, the outer cover may be divided into at least two sections. For example, the outer shell may be divided into a first section that may be in direct contact with adjacent fingers and a second section (e.g., the thermally isolated portion of the outer cover) that is exposed to the ambient air and thermally isolated from the first section. By segmenting and thermally isolating the portion of the outer shell that is in contact with the TEG components, the temperature difference between the inner shell and the second section of the outer shell may be maximized by reducing or eliminating heat from adjacent fingers from increasing the temperature of the second section of the outer shell, thereby increasing the efficiency and electrical output of the TEG components.

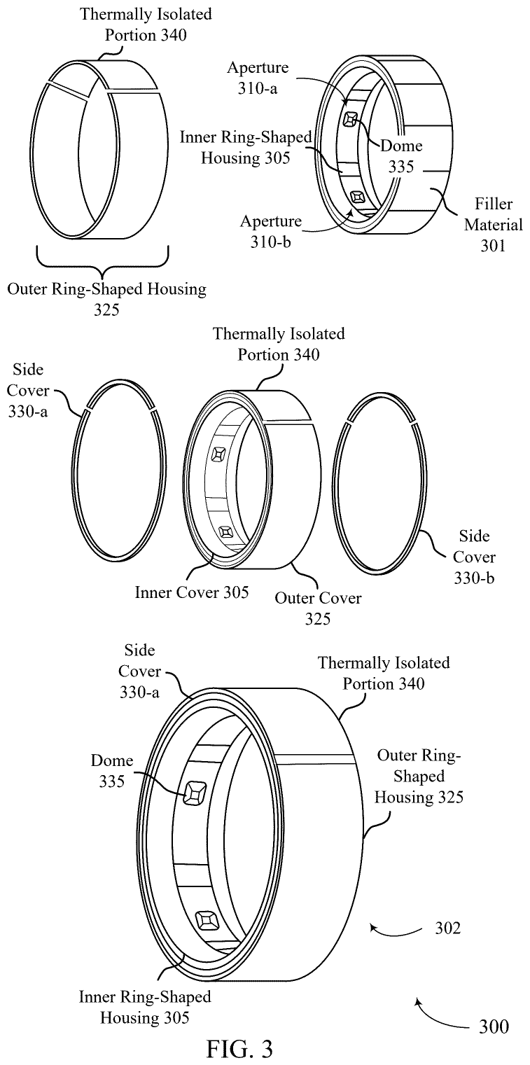

FIG. 3 shows an example of a system 300 that supports a wearable device with energy harvesting modules in accordance with aspects of the present disclosure. In particular, FIG. 3 illustrates a wearable ring device 302 that includes one or more sensors as shown and described in FIGS. 1 and 2. For example, as shown in FIG. 3, the wearable ring device 302 may include the inner ring-shaped housing 305 (e.g., inner cover, inner shell) and the outer ring-shaped housing 325 (e.g., outer cover, outer shell) described previously herein.

The inner ring-shaped housing 305 may define an inner curved surface (e.g., inner circumferential surface) of the wearable ring device 302. The wearable ring device 302 may include a filler material 301 (e.g., clear epoxy material) configured to substantially cover and seal the PCB and/or battery of the wearable ring device 302 to the inner ring-shaped housing 305. The inner ring-shaped housing 305 may include one or more apertures 310-a, 310-b that enable the sensors to collect physiological data from the user through the inner ring-shaped housing 305. In some aspects, the wearable ring device 302 may include one or more domes 335 within the inner curved surface of the inner ring-shaped housing 305 that substantially fill and/or cover the one or more apertures 310. In this regard, the domes 335 may be made of the filler material 301 that is used to seal the PCB to the inner ring-shaped housing 305. The domes 335 may exhibit any shape, such as multi-faceted domes 335 (as shown in FIG. 3), curved domes 335 (e.g., spherical or elliptical-shaped domes 335), or both.

The wearable ring device 302 may include an outer ring-shaped housing 325 (e.g., outer cover, outer shell). The outer ring-shaped housing 325 may be secured to the inner ring-shaped housing 305 using side covers 330, such a side cover 330-a and a side cover 330-b. In some aspects, the side covers 330 may include ring-shaped fittings, molded side covers (e.g., UV or heat-activated adhesive), or both. For example, the side covers 330 may include ring-shaped fittings (e.g., compression fittings) that are inserted into slots/grooves between the inner ring-shaped housing 305 and the outer ring-shaped housing 325. In other cases, the slots/grooves between the inner ring-shaped housing 305 and the outer ring-shaped housing 325 may be filled with an adhesive or filler material (e.g., UV or heat-activated adhesive, other insulation material), where the material is subsequently cured or molded to form the side covers 330.

The outer ring-shaped housing 325 may include a thermally isolated portion 340 that is thermally isolated from the remaining sections of the outer ring-shaped housing 325. For example, the outer ring-shaped housing 325 may include at least two grooves where one groove is between a first side of the thermally isolated portion 340 and the remaining portions of the outer ring-shaped housing 325, and a second groove is between a second side of the thermally isolated portion 340 and the remaining portions of the outer ring-shaped housing 325. As shown in FIG. 3, the outer ring-shaped housing 325 may include at least two sections where the first section (e.g., the thermally isolated portion 340) extends partially around the circumference (e.g., curved perimeter) of the wearable ring device 302, and the second section extends partially around the other side of circumference of the wearable ring device 302. In some cases, the grooves that separate the thermally isolated portion 340 may be filled with the same material that is used to form the side covers 330 (e.g., UV or heat-activated adhesive, other insulating material).

As described herein, the wearable ring device 302 may utilize energy-harvesting techniques integrating one or more TEG components between the inner ring-shaped housing 305 and the thermally isolated portion 340 of the outer ring-shaped housing 325. In particular, TEG components of the wearable ring device 302 may generate electricity that may be used to power the sensors and/or recharge a battery of the wearable ring device 302 based on a temperature difference between the inner ring-shaped housing 305 and the thermally isolated portion 340 of the outer ring-shaped housing 325.

The thermally isolated portion 340 may be thermally isolated from both the inner ring-shaped housing 305 and the other portions of the outer ring-shaped housing 325. In such cases, the inner ring-shaped housing 305 may be thermally isolated from the at least one thermally isolated portion 340 and the remaining portion of the outer ring-shaped housing 325. Integrating the one or more TEG components between the thermally isolated portion 340 and the inner ring-shaped housing 305 may prevent heat from adjacent fingers from warming up the portion of the outer ring-shaped housing 325 used for energy harvesting (e.g., the thermally isolated portion 340).

For example, while the wearable ring device 302 is being worn, portions of the outer ring-shaped housing 325 may contact one or more adjacent fingers on the user's hand, where heat from the adjacent fingers heats up the outer ring-shaped housing 325, thereby reducing a temperature difference between the inner and outer covers, and reducing an efficiency of the TEG components. As such, by thermally isolating the thermally isolated portion 340 of the outer ring-shaped housing 325, heating caused by adjacent fingers may be reduced or eliminated. That is, adjacent fingers may heat the remaining portion of the outer ring-shaped housing 325, but not the thermally isolated portion 340, thereby maintaining a high temperature difference between the inner ring-shaped housing 305 and the thermally isolated portion 340 that may be leveraged by TEG components for energy harvesting.

The side covers 330 may include an insulating material that is configured to insulate the outer ring-shaped housing 325, the inner ring-shaped housing 305, the electrical components, the filler material 301, or a combination hereof. The side covers 330 may each include a segmented portion that mirrors the segmented portion of the outer ring-shaped housing 325. For example, the side cover 330-a may include one or more grooves between an isolated region of the side cover 330-a and the remaining portions of the side cover 330-a. The side cover 330-b may include one or more grooves between an isolated region of the side cover 330-b and the remaining portions of the side cover 330-b. In some cases, the side covers 330 may include a continuous surface (e.g., without gaps, segmented portions, and the like) that extends around an entire circumference of the side cover 330.

In some cases, the side covers 330 may secure (e.g., couple) the outer ring-shaped housing 325 to the inner ring-shaped housing 305. For instance, the side cover 330-a (e.g., a first ring-shaped fitting) may extend around the circumference of the wearable ring device 302 on a first lateral side and the side cover 330-b (e.g., a second ring-shaped fitting) may extend around the circumference of the wearable ring device 302 on a second lateral side. Thus, the wearable ring device 302 may include the outer ring-shaped housing 325 (e.g., including the thermally isolated portion 340), the inner ring-shaped housing 305, the electrical components, the side cover 330-a, and the side cover 330-b. The outer ring-shaped housing 325 may be secured to one or more portions of the inner ring-shaped housing 305 such that there are one or more gaps between the outer ring-shaped housing 325 and the inner ring-shaped housing 305. The one or more gaps may enable communication (e.g., transmission and reception) of wireless signals into and out of the wearable ring device 302 (e.g., through the side covers 330).

In some cases, securing the side covers 330 on to a wearable ring device 302 may include pressing the side cover 330-a to the first lateral side and pressing the side cover 330-b to the second lateral side. Thus, the side cover 330-a and the side cover 330-b may lock (e.g., couple) the outer ring-shaped housing 325 to the inner ring-shaped housing 305. Additionally, or alternatively, the outer ring-shaped housing 325, the inner ring-shaped housing 305, or both, may be pressed such that the outer ring-shaped housing 325 is concentric to the inner ring-shaped housing 305. In such cases, the wearable ring device 302 may include two concentric metallic rings.

The battery life of the wearable ring device 302 may be increased by implementing energy harvesting techniques, as described herein. In some cases, energy harvesting techniques may implement solar cells, thermal energy harvesters, kinetic energy harvesters, and the like, that generate electricity directly from the environment and/or from the human body. Energy harvesting techniques described herein may utilize a temperature difference between different materials (or two points within a material) to generate an electric voltage and/or electric current. The electric voltage generated may be proportional to the temperature difference between the respective materials/components, and may depend on the materials used.

As described herein, the wearable ring device 302 may include one or more TEGs that generate a voltage across a thermocouple based on a temperature difference between the thermally isolated portion 340 and the inner ring-shaped housing 305. The voltage generated may be used to power electronic devices, charge a battery of the wearable ring device 302, or both. The wearable ring device 302 may collect physiological data associated with the user and generate electrical signals for use in energy harvesting. For example, the wearable ring device 302 may collect physiological data associated with a user via one or more sensors of the wearable ring device 302 and generate, using the TEG components, an electric current based on a temperature difference between the inner ring-shaped housing 305 and the at least one thermally isolated portion 340 of the outer ring-shaped housing 325.

The temperature difference between the inner ring-shaped housing 305 and the thermally isolated portion 340 of the outer ring-shaped housing 325 may be achieved based on temperature differences caused between the body heat of the user and the surrounding environment. For example, the inner ring-shaped housing 305 may be heated based on the inner ring-shaped housing 305 contacting a tissue of the user. Comparatively, the thermally isolated portion 340 of the outer ring-shaped housing 325 may be cooled based on the thermally isolated portion 340 being exposed to a surrounding environment of the user, thereby creating a temperature difference between the inner ring-shaped housing 305 and the thermally isolated portion 340.

In some cases, the wearable ring device 302 may include additional features to help cool the outer ring-shaped housing 325 and/or help heat the inner ring-shaped housing 305, and thereby increase the temperature difference that is used for energy harvesting. For example, the inner ring-shaped housing 305, the thermally isolated portion 340, or both, may include a textured surface that is configured to increase the temperature difference between the inner ring-shaped housing 305 and the thermally isolated portion 340 of the outer ring-shaped housing 325 by increasing a surface area of the inner ring-shaped housing 305, the thermally isolated portion 340, or both. In such cases, the inner ring-shaped housing 305 may include a textured surface on an inside surface of the inner ring-shaped housing 305 that contacts the skin surface of the user and is configured to help heat the inner ring-shaped housing 305. The thermally isolated portion 340 may include a textured surface on the outside surface of the thermally isolated portion 340 that is exposed to the ambient environment and is configured to cool the outer ring-shaped housing 325. In some cases, the textured surface on the outside surface of the thermally isolated portion 340 may be configured to capture and maintain liquid that may be evaporated, thereby cooling the surface of the thermally isolated portion 340.

In other cases, heat-generating components of the wearable ring device 302, such as processors, sensors, the battery, etc.), may be placed against the inner ring-shaped housing 305 to increase the temperature of the inner ring-shaped housing 305, and thereby increase the temperature difference between the inner ring-shaped housing 305 and the thermally isolated portion 340 that is used for energy harvesting.

The electric current generated by the TEG components of the wearable ring device 302 may be used to recharge a battery of the wearable ring device 302, power the one or more sensors, or both. In some cases, the user may be able to increase the amount of power/current provided by the TEG components by placing the wearable ring device 302 in contact with a cool surface, increasing their body temperature, or both. For example, the wearable ring device 302 may be placed in contact with the charging device that is at a lower temperature than the wearable ring device 302, thereby cooling the outer surface of at least the thermally isolated portion 340. In such cases, the charging device may charge the wearable ring device 302 with increased efficiency by cooling the energy harvesting portion of the outer ring-shaped housing 325 (e.g., the thermally isolated portion 340).

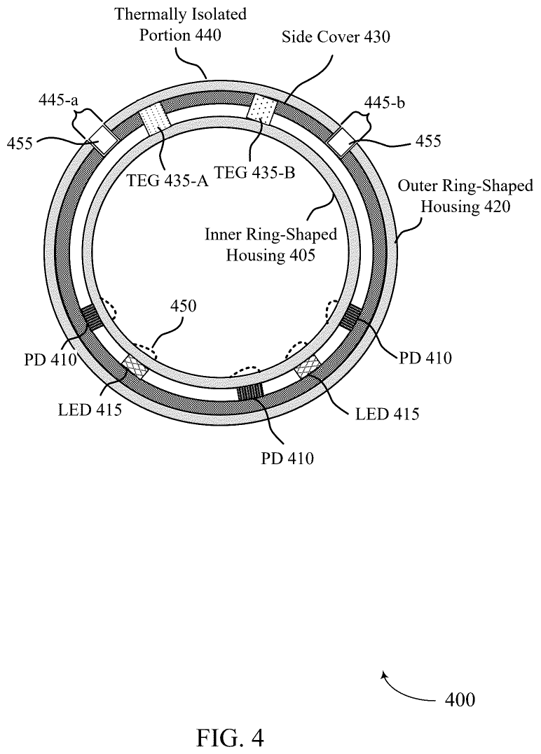

FIG. 4 shows an example of a wearable ring device 400 that includes energy harvesting module in accordance with aspects of the present disclosure. The wearable ring device 400 may implement, or be implemented by, aspects of the system 100, the system 200, the system 300, or any combination thereof. For example, the wearable ring device 400 may be an example of wearable devices as described with reference to FIGS. 1 through 3. As an illustrative example, the wearable ring device 400 shown and described in FIG. 4 may include a cross sectional view of the wearable ring device 302 shown and described in FIG. 3. Although the wearable devices are illustrated as being circular in FIG. 4, it is contemplated herein that the energy-harvesting techniques described herein may be implemented in wearable devices associated with any shape and any type of a wearable device (e.g., a ring, a watch or wristband, an armband, a necklace, and the like).

As described previously herein, the wearable ring device 400 may include at least an inner ring-shaped housing 405 (e.g., inner ring-shaped housing), one or more electrical components (e.g., one or more PDs 410, one or more LEDs 315, etc.), an outer ring-shaped housing 420 (e.g., outer ring-shaped housing), an epoxy layer (e.g., filler material 301), and one or more side covers 430. In some cases, the wearable ring device 400 may include an electronic substrate, such as a printed wiring board (PWB) or PCB. The electronic substrate may be attached (e.g., coupled) to the inner ring-shaped housing 405. The inner ring-shaped housing 405 may include an inner metallic shell and may include one or more apertures. In such cases, the respective electronic substrates may include or may be connected (e.g., communicatively coupled) to sensors disposed on/within the inner ring-shaped housing 405.

For the purposes of the present disclosure, the term “sensor” may be used to refer to a module including a pair of light-emitting and light-receiving components, such as one or more LEDs 415 and one or more PDs 410. Moreover, the light-emitting component and light-receiving component of a “sensor” may be co-located (e.g., positioned within the same sensor housing) and/or may be positioned at different locations within the wearable ring device 400. Additionally, in some cases, a “sensor” may include other components in addition to the LEDs 415 and the PDs 410, such as lenses.

In some implementations, the wearable ring device 400 may include domes 450 over the optical components (e.g., LEDs 415, PDs 410) of the inner ring-shaped housing 405. That is, the LEDs 415 and PDs 410 may be disposed within, or otherwise aligned with, apertures within the inner ring-shaped housing 405, where the domes 450 may substantially cover and/or fill the apertures. It has been found that such domes 450 that cover the apertures may result in better contact with a tissue of the user's skin, thereby improving the quality of collected physiological data collected by the wearable ring device 400. The domes 450 may be formed to be curved (e.g., spherical-shaped, elliptical-shaped), multi-faceted, or both.

The wearable ring device 400 may include the outer ring-shaped housing 420. The outer ring-shaped housing 420 may include an outer metallic shell that is a same or different material as compared to the inner ring-shaped housing 405. For instance, the inner ring-shaped housing 405 may be manufactured from titanium (e.g., a titanium inlet ring), while the outer ring-shaped housing 420 may be manufactured from steel. It is contemplated herein that the inner ring-shaped housing 405 and/or the outer ring-shaped housing 420 may be manufactured of any thermally-conductive materials. Moreover, in some cases, the thermally-isolated portion 440 of the outer cover may include a metallic or other thermally-conductive material, where the remaining portion of the outer ring-shaped housing 420 may be manufactured of a different material (e.g., non-thermally-conductive material). In some cases, side covers 430 may be used to secure the outer ring-shaped housing 420 to the inner ring-shaped housing 405. For example, a first side cover 430 and a second side cover 430 may fill gaps between the outer ring-shaped housing 420 and the inner ring-shaped housing 405 on a first lateral side and a second lateral side of the wearable ring device, respectively.

In some cases, the wearable ring device 400 may use additional power during operation such as during exercise when heart rate measurements may be made more frequently and due to the motion artifacts using more power than the LEDs 415. However, there may be limited space in the wearable ring device 400 for available battery capacity. In such cases, the wearable ring device 400 may implement energy harvesting techniques by harvesting energy based on a temperature difference between two concentric metallic rings and using at least one TEG component 435 between the two concentric rings (e.g., the inner ring-shaped housing 405 and the outer ring-shaped housing 420).

As shown in FIG. 4, the wearable ring device 400 may include a first TEG component 435-a and a second TEG component 435-b. The TEG components 435 may extend from an inner surface of the thermally isolated portion 440 of the outer ring-shaped housing 420 to an inner surface of the inner ring-shaped housing 405. In such cases, the TEG components 435 may extend through the side cover 430, the epoxy layer (e.g., filler material 401), the PCB, or a combination thereof. The TEG components 435 may be arranged in a series configuration along the thermally isolated portion 440. In some cases, the first TEG component 435-a the second TEG component 435-b may be in contact with each of the thermally isolated portion 440 of inner ring-shaped housing 405 and the outer ring-shaped housing 420, thereby enabling heat transfer between/across the thermally isolated portion 440, the TEG components 435, and the inner ring-shaped housing 405. In some cases, the wearable ring device 400 may include more than one thermally isolated portion 440 that is used for energy harvesting. In such cases, each thermally isolated portion 440 may include at least one TEG component 435 coupled with the thermally isolated portion 440.

As described previously herein, the one or more TEG components 435 may be configured to generate power/electricity based on a temperature difference between the inner ring-shaped housing 405 and the thermally-isolated portion 440 of the outer ring-shaped housing 420. In particular, heat from the user's tissue may heat the inner ring-shaped housing 405, and the surrounding environment (e.g., ambient air) may cool the thermally isolated portion 440 of the outer ring-shaped housing 420, thereby creating a temperature difference that is used by the TEG components 435 for energy harvesting.

In some aspects, various techniques may be used to increase the temperature difference between the inner ring-shaped housing 405 and the thermally-isolated portion 440 of the outer ring-shaped housing 420, such as by heating the inner ring-shaped housing 405 and/or cooling the outer ring-shaped housing 420.

For example, in some cases, the heat-generating portions (e.g., LEDs 415 and/or PDs 410) may be positioned against the inner ring-shaped housing 405 to increase the temperature of the inner ring-shaped housing 405, thereby increasing the temperature difference used for energy harvesting. For instance, the one or more sensors, one or more processors of the wearable ring device 400, or both, may be positioned against the inner ring-shaped housing 405 such that heat generated by the one or more sensors, one or more processors, or both heats up the inner ring-shaped housing 405 to increase the temperature difference between the inner ring-shaped housing 405 and the outer ring-shaped housing 420, including the thermally isolated portion 440. In such cases, the electronics of the wearable ring device 400 that generate heat may be placed closely to the inlet sensors to increase the temperature difference between the inner ring-shaped housing 405 and the thermally isolated portion 440 of the outer ring-shaped housing 420, and thereby increase the output/efficiency of the TEG components 435.

In some aspects, the LEDs 415, PDs 410, or both, may be positioned opposite of the TEG components 435 and/or the thermally isolated portion 440 such that the LEDs 415 and PDs 410 are on the palm-side, and the thermally isolated portion 440 is on the top-side of the wearable ring device 400 such that the thermally isolated portion 440 is exposed to ambient air. For example, the one or more sensors may be positioned within a first radial span of a circumference (e.g., curved perimeter) of the wearable ring device 400 that is radially opposite of the thermally isolated portion 440 of the outer ring-shaped housing 420. This specific configuration or arrangement of the LEDs 415 and PDs 410 with respect to the thermally isolated portion 440 (e.g., including the TEG components 435) may ensure that the thermally isolated portion 440 is exposed to the environment and is thermally isolated from the user's adjacent fingers (which would otherwise heat the thermally-isolated portion 440, reduce the temperature difference, and decrease the output/efficiency of the TEG components 435).

In other words, the one or more sensors (e.g., including the LEDs 415 and the PDs 410) and the thermally isolated portion 440 may be positioned around a circumference of the wearable ring device 400 such that, when the one or more sensors are positioned on a palm-side of a finger of a user, the thermally isolated portion 440 is positioned on a dorsal-side of the finger, and the remaining portion of the outer ring-shaped housing 420 is adjacent to one or more additional fingers that are adjacent to the finger of the user. In such cases, the inner ring-shaped housing 405 may be heated from body heat (e.g., contact with the finger of the user), the remaining portion of the outer ring-shaped housing 420 may be heated by body heat (e.g., via contact with one or more additional fingers that are adjacent to the finger of the user), and the thermally isolated portion 440 may be cooled by ambient air and not heated by contact with either the finger wearing the wearable ring device 400 or adjacent fingers.

In some examples, the thermally isolated portion 440 of the outer ring-shaped housing 420 (e.g., a first section) may be positioned on the palm-side of the finger of the user, and a second of the outer ring-shaped housing 420 may be positioned on the dorsal-side of the finger that is exposed to ambient air. In such cases, the thermally isolated portion 440 may be cooled or heated by positing the hand in contact with a cold or hot object, respectively, thereby increasing the temperature difference between the thermally isolated portion 440 and the remaining sections of the outer ring-shaped housing 420. The wearable ring device 400 (and/or corresponding user device 106) may be able to instruct the user to rotate the wearable ring device 400 to change the positioning of the thermally isolated portion 440 from the dorsal-side of the finger to the palm-side of the finger and vice versa. For example, the wearable ring device 400 may transmit an instruction (e.g., audio message, visual message, and the like) to the user to rotate the wearable ring device 400 so that the user can easily place their palm (and the ring) against a cold object (e.g., dedicated cold charger, cold water bottle, etc.) for energy-harvesting.

The outer ring-shaped housing 420 may include one or more grooves 445 to create the thermally isolated portion 440. The grooves 445 may be disposed within the outer ring-shaped housing 420 and extend through an entire width of the outer ring-shaped housing 420. In some cases, the grooves 445 may extend through an entire width of the side covers 430. The grooves 445 may be filled with a thermally-insulating material 455 that is configured to thermally isolate the thermally isolated portion 440 from the remaining portion of the outer ring-shaped housing 420. The use of the thermally-insulating material 455 (e.g., via the grooves 445, the side covers 430, or both) may increase the efficiency of the TEG component 435 may prevent the thermally-isolated portion 440 from being heated by the remainder of the outer ring-shaped housing 420 that is in contact with adjacent fingers of the user's hand.

In some cases, the wearable ring device 400 may include pockets of phase change material between the inner ring-shaped housing 405 and outer ring-shaped housing 420. The phase change material may be configured to store heat through changing physical states (e.g., transitioning from solid to liquid) and release the stored heat at a later time. For example, the wearable ring device 400 may include one or more reservoirs positioned between the outer ring-shaped housing 420 and the inner ring-shaped housing 405, where the one or more reservoirs are at least partially filled with the phase change material. In some cases, the reservoirs including the phase change material may be positioned between the inner ring-shaped housing 405 and the thermally isolated portion 440 of the outer ring-shaped housing 420. In such cases, the reservoirs may be positioned adjacent to the TEG components 435.

A change of the phase change material from a first phase to a second phase may be configured to store energy from the electric current generated by the TEG components 435, generate an additional electric current to power the one or more sensors, recharge the battery of the wearable ring device, or both. In some cases, the wearable ring device 400 may store energy during the charging of the wearable ring device 400 by causing a phase change within stored reservoirs of phase change material, where the stored energy may later be extracted/used by causing an inverse phase change in the phase change material.

In some cases, the TEG components 435 may require a small amount of energy/power in order to perform energy-harvesting. That is, the TEG components 435 may not be completely passive. For example, a voltage/current output by the TEG components 435 may have to be converted into a usable voltage/current level in order to be used by the ring, where the conversion of the voltage/current requires some amount of energy expenditure. Accordingly, to save energy and power, the TEG components 435 may be activated based on one or more trigger conditions. That is, the wearable ring device 400 may implement different trigger conditions to activate the TEG components 435 to ensure that energy created by the TEG components 435 will offset any energy that is used to activate the TEG components 435 and/or convert a voltage/current output by the TEG components 435 (e.g., trigger conditions implemented to ensure that use of TEG components 435 results in a net-positive energy/power gain).

For example, the TEG components 435 may be activated based on sensing temperature, sensing motion, sensing voltage, or a combination thereof. For example, the TEG components 435 may be activated based on (e.g., after) collecting the physiological data via the one or more sensors of the wearable ring device 400. The physiological data includes at least temperature data, motion data, or both. In such cases, generating the electric current is based on activating the one or more TEG components 435.

The wearable ring device 400 may include temperature sensors to sense the temperature differential between the inner ring-shaped housing 405 and the thermally isolated portion 440, and activate the TEG components 435 once the temperature difference exceeds a threshold (e.g., is sufficiently high). For example, the wearable ring device 400 may include one or more processors that are communicatively coupled with the one or more sensors (e.g., temperature sensors) and the TEG components 435. The one or more processors may be configured to receive temperature data acquired by the one or more temperature sensors and determine the temperature difference between the inner ring-shaped housing 405 and the thermally isolated portion 440 of the outer ring-shaped housing 420 based on the temperature data. For instance, the wearable ring device 400 may include separate temperature sensors disposed on/within the inner ring-shaped housing 405, the outer ring-shaped housing 420, the thermally isolated portion 440, or any combination thereof. In such cases, the one or more processors may activate the TEG components 435 based on the temperature difference between the inner ring-shaped housing 405 and the thermally isolated portion 440 satisfying a threshold temperature difference.

In some cases, the wearable ring device 400 may include motion sensors (e.g., activity sensors) to sense motion and activate the TEG components 435 once the sensed motion exceeds a threshold. Increased motion may be associated with an increase in skin temperature, an increase in air movement across the surface of the outer ring-shaped housing 420 to cool the outer ring-shaped housing 420, or both. For example, as the user goes on a run, their skin temperature may increase (thereby increasing the temperature of the inner ring-shaped housing 405), and swinging arm motion during the run may result in increased air flow on the ring (thereby cooling the outer ring-shaped housing 420). As such, motion of the user may be used as a proxy for estimating a temperature difference within the ring, and may thereby be used to activate the TEG components 435 for energy harvesting.

For example, the wearable ring device 400 may include one or more processors that are communicatively coupled with the one or more sensors (e.g., motion sensors) and the TEG components 435. The one or more processors may be configured to receive motion data acquired by the one or more motion sensors and activate the TEG components 435 based on the motion data satisfying a threshold motion level.

In some examples, the TEG components 435 may be activated based on a voltage of the TEG components 435. In particular, a voltage/current output by the TEG components 435 may be used to estimate the temperature difference, and thereby estimate a voltage/current that will be generated by the TEG components 435. For example, the wearable ring device 400 may monitor the output of the TEG components 435 to determine when to activate energy-harvesting components (e.g., the TEG components 435). The one or more processors may be configured to monitor a voltage, a current level, or both, associated with the TEG components 435 and activate the TEG components 435 based on the voltage, the current level, or both, satisfying one or more thresholds.

Monitoring whether the voltage from the TEG components 435 reaches a threshold may indicate to the wearable ring device 400 to use other components to turn the voltage into a usable voltage (e.g., a voltage that supplies power to at least the battery of the wearable ring device 400). For example, the wearable ring device 400 may determine that the temperature difference between the inner ring-shaped housing 405 and the thermally isolated portion 440 of the outer ring-shaped housing 420 satisfies a threshold. In such cases, the wearable ring device 400 may convert a voltage outputted by the TEG components 435 to a usable voltage associated with the electric current based on the temperature difference satisfying the threshold. In such cases, the system may refrain from activating the TEG components 435 if the temperature difference fails to satisfy a threshold (e.g., is below the threshold), thereby saving power and increasing the efficiency of the wearable ring device 400.

In some cases, the wearable ring device 400 may calibrate skin temperature measurements based on the voltage/current output by the TEG components 435. In particular, the voltage/current output by the TEG components 435 may be used to estimate the temperature difference across the inner/outer covers, and thereby estimate an effect that the temperature of the surrounding environment has on collected skin temperature measurements. Based on monitoring the voltage, current level, or both output by the TEG components 435, the wearable ring device 400 may estimate an additional temperature difference between a skin temperature of the user and a surrounding environment of the user based on the voltage, the current level, or both. In such cases, the wearable ring device 400 may calibrate one or more skin temperature measurements received from the one or more temperature sensors based on the additional temperature difference. The wearable ring device 400 may use the electric current to calibrate skin temperature measurements.

A stress analysis feature of the wearable ring device 400 may rely on skin temperature measurements. As skin temperature mirrors the ambient temperature due to vasculature accommodation (e.g., vasoconstriction and vasodilation), reliable skin temperature measurement may use ambient temperature as an input for the stress analysis feature. In such cases, the same segmented ring structure (e.g., including the thermally isolated portion 440) that may be used for energy harvesting may also be used for the stress analysis feature as current generated over the TEG component 435 may be used for ambient air temperature assessment.

In general, the wearable device structure including TEG components 435 may be used to estimate a difference between the user's skin temperature and the surrounding ambient air temperature. Such temperature estimations may be used to perform or otherwise improve any physiological measurements that rely on skin temperature. For example, in some cases, menstrual cycle detection and/or estimation may rely on skin temperature measurements, which may be performed using outputs from the TEG components 435, as described herein. In such cases, the same segmented ring structure (e.g., including the thermally isolated portion 440) that may be used for energy harvesting may also be used for a menstrual cycle estimation feature as current generated over the TEG component 435 may be used for ambient air temperature assessment. In some examples, the wearable ring device 400 may use the segmented ring structure (e.g., including the thermally isolated portion 440) to track sleeping environment conditions (e.g., temperature, humidity, air flow, etc.) and provide feedback to the user whether the temperature of the ambient air increases or decreases the quality of the user's sleep. In other examples, the wearable ring device 400 may use the thermally isolated portion 440 to determine a correlation between the temperature of the ambient air and the user's heart rate and provide feedback to the user how the temperature of the ambient air affects a user's activity performance.

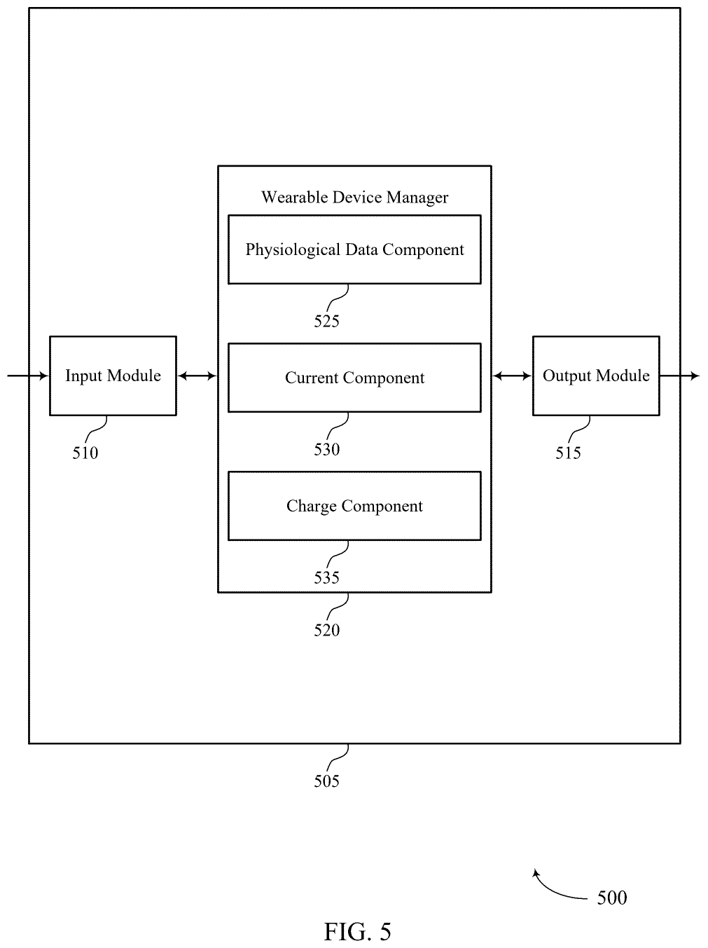

FIG. 5 shows a block diagram 500 of a device 505 that includes energy harvesting modules in accordance with aspects of the present disclosure. The device 505 may include an input module 510, an output module 515, and a wearable device manager 520. The device 505, or one or more components of the device 505 (e.g., the input module 510, the output module 515, and the wearable device manager 520), may include at least one processor, which may be coupled with at least one memory, to support the described techniques. Each of these components may be in communication with one another (e.g., via one or more buses).

For example, the wearable device manager 520 may include a physiological data component 525, a current component 530, a charge component 535, or any combination thereof. In some examples, the wearable device manager 520, or various components thereof, may be configured to perform various operations (e.g., receiving, monitoring, transmitting) using or otherwise in cooperation with the input module 510, the output module 515, or both. For example, the wearable device manager 520 may receive information from the input module 510, send information to the output module 515, or be integrated in combination with the input module 510, the output module 515, or both to receive information, transmit information, or perform various other operations as described herein.

The wearable device manager 520 may support operating a wearable ring device in accordance with examples as disclosed herein. The physiological data component 525 may be configured as or otherwise support a means for collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing. The current component 530 may be configured as or otherwise support a means for generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing. The charge component 535 may be configured as or otherwise support a means for recharging a battery of the wearable ring device, powering the one or more sensors, or both, using the electric current.

The physiological data component 525 may be configured as or otherwise support a means for activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

The current component 530 may be configured as or otherwise support a means for determining that the temperature difference between the inner ring-shaped housing and the at least one thermally isolated portion of the outer ring-shaped housing satisfies a threshold. The current component 530 may be configured as or otherwise support a means for converting a voltage outputted by the one or more TEG components to a usable voltage associated with the electric current based at least in part on the temperature difference satisfying the threshold.

The physiological data component 525 may be configured as or otherwise support a means for receiving temperature data acquired by the one or more sensors. The physiological data component 525 may be configured as or otherwise support a means for determining the temperature difference between the inner ring-shaped housing and the at least one thermally isolated portion of the outer ring-shaped housing based at least in part on the temperature data. The physiological data component 525 may be configured as or otherwise support a means for activating the one or more TEG components based at least in part on the temperature difference satisfying a threshold temperature difference, wherein generating the electric current is based at least in part on activating the one or more TEG components.

The physiological data component 525 may be configured as or otherwise support a means for receiving motion data acquired by the one or more sensors. The physiological data component 525 may be configured as or otherwise support a means for activating the one or more TEG components based at least in part on the motion data satisfying a threshold motion level, wherein generating the electric current is based at least in part on activating the one or more TEG components.

The current component 530 may be configured as or otherwise support a means for monitoring a voltage, a current level, or both, associated with the one or more TEG components. The current component 530 may be configured as or otherwise support a means for activating the one or more TEG components based at least in part on the voltage, the current level, or both, satisfying one or more thresholds, wherein generating the electric current is based at least in part on activating the one or more TEG components.

The current component 530 may be configured as or otherwise support a means for monitoring a voltage, a current level, or both, associated with the one or more TEG components. The physiological data component 525 may be configured as or otherwise support a means for estimate an additional temperature difference between a skin temperature of the user and a surrounding environment of the user based at least in part on the voltage, the current level, or both. The physiological data component 525 may be configured as or otherwise support a means for calibrating one or more skin temperature measurements received from the one or more temperature sensors based at least in part on the additional temperature difference.

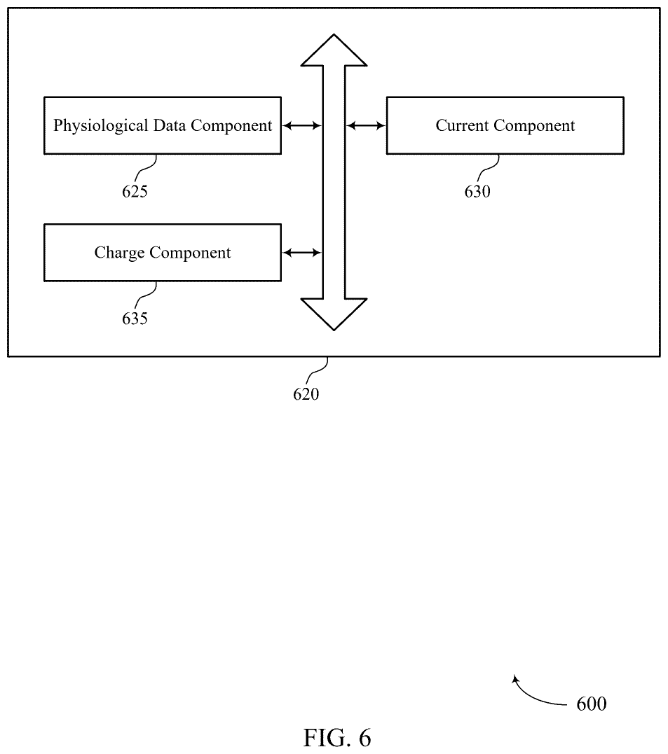

FIG. 6 shows a block diagram 600 of a wearable device manager 620 that supports a wearable device with energy harvesting modules in accordance with aspects of the present disclosure. The wearable device manager 620 may be an example of aspects of a wearable device manager or a wearable device manager 520, or both, as described herein. The wearable device manager 620, or various components thereof, may be an example of means for performing various aspects of wearable device with energy harvesting module as described herein. For example, the wearable device manager 620 may include a physiological data component 625, a current component 630, a charge component 635, or any combination thereof. Each of these components, or components of subcomponents thereof (e.g., one or more processors, one or more memories), may communicate, directly or indirectly, with one another (e.g., via one or more buses).

The wearable device manager 620 may support operating a wearable ring device in accordance with examples as disclosed herein. The physiological data component 625 may be configured as or otherwise support a means for collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing. The current component 630 may be configured as or otherwise support a means for generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing. The charge component 635 may be configured as or otherwise support a means for recharging a battery of the wearable ring device, powering the one or more sensors, or both, using the electric current.

In some examples, the physiological data component 625 may be configured as or otherwise support a means for activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

In some examples, the current component 630 may be configured as or otherwise support a means for determining that the temperature difference between the inner ring-shaped housing and the at least one thermally isolated portion of the outer ring-shaped housing satisfies a threshold. In some examples, the current component 630 may be configured as or otherwise support a means for converting a voltage outputted by the one or more TEG components to a usable voltage associated with the electric current based at least in part on the temperature difference satisfying the threshold.

In some examples, the physiological data component 625 may be configured as or otherwise support a means for receiving temperature data acquired by the one or more sensors. In some examples, the physiological data component 625 may be configured as or otherwise support a means for determining the temperature difference between the inner ring-shaped housing and the at least one thermally isolated portion of the outer ring-shaped housing based at least in part on the temperature data. In some examples, the physiological data component 625 may be configured as or otherwise support a means for activating the one or more TEG components based at least in part on the temperature difference satisfying a threshold temperature difference, wherein generating the electric current is based at least in part on activating the one or more TEG components.

In some examples, the physiological data component 625 may be configured as or otherwise support a means for receiving motion data acquired by the one or more sensors. In some examples, the physiological data component 625 may be configured as or otherwise support a means for activating the one or more TEG components based at least in part on the motion data satisfying a threshold motion level, wherein generating the electric current is based at least in part on activating the one or more TEG components.

In some examples, the current component 630 may be configured as or otherwise support a means for monitoring a voltage, a current level, or both, associated with the one or more TEG components. In some examples, the current component 630 may be configured as or otherwise support a means for activating the one or more TEG components based at least in part on the voltage, the current level, or both, satisfying one or more thresholds, wherein generating the electric current is based at least in part on activating the one or more TEG components.

In some examples, the current component 630 may be configured as or otherwise support a means for monitoring a voltage, a current level, or both, associated with the one or more TEG components. In some examples, the physiological data component 625 may be configured as or otherwise support a means for estimate an additional temperature difference between a skin temperature of the user and a surrounding environment of the user based at least in part on the voltage, the current level, or both. In some examples, the physiological data component 625 may be configured as or otherwise support a means for calibrating one or more skin temperature measurements received from the one or more temperature sensors based at least in part on the additional temperature difference.

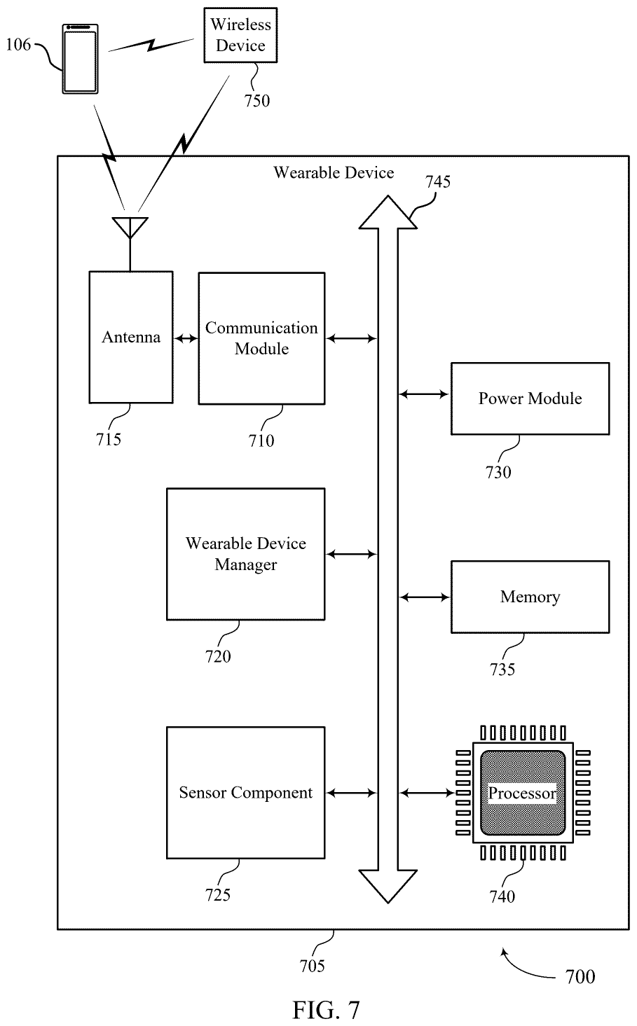

FIG. 7 shows a diagram of a system 700 including a device 705 that supports wearable devices with energy harvesting modules in accordance with aspects of the present disclosure. The device 705 may be an example of or include the components of a device 505 as described herein. The device 705 may include an example of a wearable device 104, as described previously herein. The device 705 may include components for bi-directional communications including components for transmitting and receiving communications with a user device 106 and a server 110, such as a wearable device manager 720, a communication module 710, an antenna 715, a sensor component 725, a power module 730, at least one memory 735, at least one processor 740, and a wireless device 750. These components may be in electronic communication or otherwise coupled (e.g., operatively, communicatively, functionally, electronically, electrically) via one or more buses (e.g., a bus 745).

The wearable device manager 720 may support operating a wearable ring device in accordance with examples as disclosed herein. For example, the wearable device manager 720 may be configured as or otherwise support a means for collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing. The wearable device manager 720 may be configured as or otherwise support a means for generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing. The wearable device manager 720 may be configured as or otherwise support a means for recharging a battery of the wearable ring device, powering the one or more sensors, or both, using the electric current.

For example, the wearable device manager 720 may be configured as or otherwise support a means for activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

For example, the wearable device manager 720 may be configured as or otherwise support a means for determining that the temperature difference between the inner ring-shaped housing and the at least one thermally isolated portion of the outer ring-shaped housing satisfies a threshold. The wearable device manager 720 may be configured as or otherwise support a means for converting a voltage outputted by the one or more TEG components to a usable voltage associated with the electric current based at least in part on the temperature difference satisfying the threshold.

For example, the wearable device manager 720 may be configured as or otherwise support a means for receiving temperature data acquired by the one or more sensors. The wearable device manager 720 may be configured as or otherwise support a means for determining the temperature difference between the inner ring-shaped housing and the at least one thermally isolated portion of the outer ring-shaped housing based at least in part on the temperature data. The wearable device manager 720 may be configured as or otherwise support a means for activating the one or more TEG components based at least in part on the temperature difference satisfying a threshold temperature difference, wherein generating the electric current is based at least in part on activating the one or more TEG components.

For example, the wearable device manager 720 may be configured as or otherwise support a means for receiving motion data acquired by the one or more sensors. The wearable device manager 720 may be configured as or otherwise support a means for activating the one or more TEG components based at least in part on the motion data satisfying a threshold motion level, wherein generating the electric current is based at least in part on activating the one or more TEG components.

For example, the wearable device manager 720 may be configured as or otherwise support a means for monitoring a voltage, a current level, or both, associated with the one or more TEG components. The wearable device manager 720 may be configured as or otherwise support a means for activating the one or more TEG components based at least in part on the voltage, the current level, or both, satisfying one or more thresholds, wherein generating the electric current is based at least in part on activating the one or more TEG components.

For example, the wearable device manager 720 may be configured as or otherwise support a means for monitoring a voltage, a current level, or both, associated with the one or more TEG components. The wearable device manager 720 may be configured as or otherwise support a means for estimating an additional temperature difference between a skin temperature of the user and a surrounding environment of the user based at least in part on the voltage, the current level, or both. The wearable device manager 720 may be configured as or otherwise support a means for calibrating one or more skin temperature measurements received from the one or more temperature sensors based at least in part on the additional temperature difference.

By including or configuring the wearable device manager 720 in accordance with examples as described herein, the device 705 may support techniques for improved communication reliability, reduced latency, improved user experience related to reduced processing, reduced power consumption, more efficient utilization of communication resources, improved coordination between devices, longer battery life, improved utilization of processing capability, and the like.

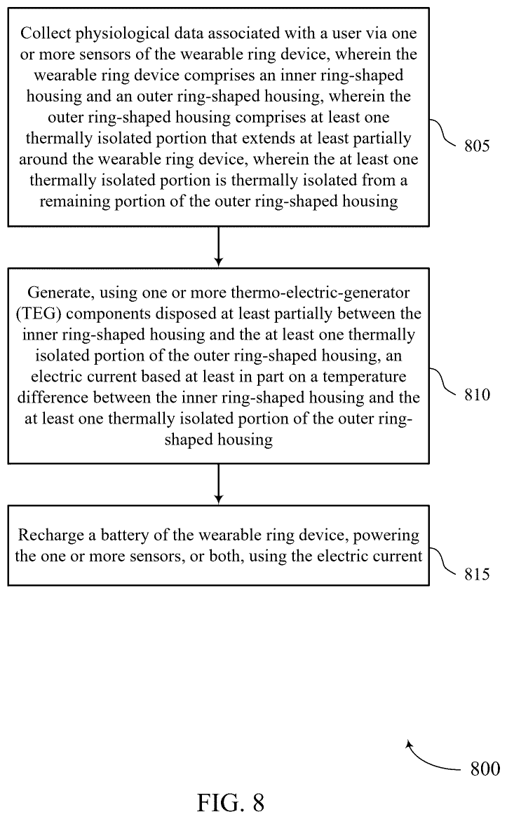

FIG. 8 shows a flowchart illustrating a method 800 that supports wearable devices with energy harvesting modules in accordance with aspects of the present disclosure. The operations of the method 800 may be implemented by a wearable device or its components as described herein. For example, the operations of the method 800 may be performed by a wearable device as described with reference to FIGS. 1 through 7. In some examples, a wearable device may execute a set of instructions to control the functional elements of the wearable device to perform the described functions. Additionally, or alternatively, the wearable device may perform aspects of the described functions using special-purpose hardware.

At 805, the method may include collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing. The operations of block 805 may be performed in accordance with examples as disclosed herein. In some examples, aspects of the operations of 805 may be performed by a physiological data component 625 as described with reference to FIG. 6.

At 810, the method may include generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing. The operations of block 810 may be performed in accordance with examples as disclosed herein. In some examples, aspects of the operations of 810 may be performed by a current component 630 as described with reference to FIG. 6.

At 815, the method may include recharging a battery of the wearable ring device, powering the one or more sensors, or both, using the electric current. The operations of block 815 may be performed in accordance with examples as disclosed herein. In some examples, aspects of the operations of 815 may be performed by a charge component 635 as described with reference to FIG. 6.

It should be noted that the methods described above describe possible implementations, and that the operations and the steps may be rearranged or otherwise modified and that other implementations are possible. Furthermore, aspects from two or more of the methods may be combined.

A method for operating a wearable ring device by an apparatus is described. The method may include collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing, generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing, and recharging a battery of the wearable ring device, powering the one or more sensors, or both, using the electric current.

An apparatus for operating a wearable ring device is described. The apparatus may include one or more memories storing processor executable code, and one or more processors coupled with the one or more memories. The one or more processors may individually or collectively operable to execute the code to cause the apparatus to collect physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing, generate, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing, and recharge a battery of the wearable ring device, powering the one or more sensors, or both, using the electric current.

Another apparatus for operating a wearable ring device is described. The apparatus may include means for collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing, means for generating, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing, and means for recharging a battery of the wearable ring device, powering the one or more sensors, or both, using the electric current.

A non-transitory computer-readable medium storing code for operating a wearable ring device is described. The code may include instructions executable by one or more processors to collect physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing, generate, using one or more TEG components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing, and recharge a battery of the wearable ring device, powering the one or more sensors, or both, using the electric current.

A method by an apparatus is described. The method may include activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

An apparatus is described. The apparatus may include one or more memories storing processor executable code, and one or more processors coupled with the one or more memories. The one or more processors may individually or collectively operable to execute the code to cause the apparatus to activate the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

Another apparatus is described. The apparatus may include means for activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

A non-transitory computer-readable medium storing code is described. The code may include instructions executable by one or more processors to activate the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

A method by an apparatus is described. The method may include determining that the temperature difference between the inner ring-shaped housing and the at least one thermally isolated portion of the outer ring-shaped housing satisfies a threshold and converting a voltage outputted by the one or more TEG components to a usable voltage associated with the electric current based at least in part on the temperature difference satisfying the threshold.

An apparatus is described. The apparatus may include one or more memories storing processor executable code, and one or more processors coupled with the one or more memories. The one or more processors may individually or collectively operable to execute the code to cause the apparatus to determine that the temperature difference between the inner ring-shaped housing and the at least one thermally isolated portion of the outer ring-shaped housing satisfies a threshold and convert a voltage outputted by the one or more TEG components to a usable voltage associated with the electric current based at least in part on the temperature difference satisfying the threshold.

Another apparatus is described. The apparatus may include means for determining that the temperature difference between the inner ring-shaped housing and the at least one thermally isolated portion of the outer ring-shaped housing satisfies a threshold and means for converting a voltage outputted by the one or more TEG components to a usable voltage associated with the electric current based at least in part on the temperature difference satisfying the threshold.

A non-transitory computer-readable medium storing code is described. The code may include instructions executable by one or more processors to determine that the temperature difference between the inner ring-shaped housing and the at least one thermally isolated portion of the outer ring-shaped housing satisfies a threshold and convert a voltage outputted by the one or more TEG components to a usable voltage associated with the electric current based at least in part on the temperature difference satisfying the threshold.

A method by an apparatus is described. The method may include receiving temperature data acquired by the one or more sensors, determining the temperature difference between the inner ring-shaped housing and the at least one thermally isolated portion of the outer ring-shaped housing based at least in part on the temperature data, and activating the one or more TEG components based at least in part on the temperature difference satisfying a threshold temperature difference, wherein generating the electric current is based at least in part on activating the one or more TEG components.

An apparatus is described. The apparatus may include one or more memories storing processor executable code, and one or more processors coupled with the one or more memories. The one or more processors may individually or collectively operable to execute the code to cause the apparatus to receive temperature data acquired by the one or more sensors, determine the temperature difference between the inner ring-shaped housing and the at least one thermally isolated portion of the outer ring-shaped housing based at least in part on the temperature data, and activate the one or more TEG components based at least in part on the temperature difference satisfying a threshold temperature difference, wherein generating the electric current is based at least in part on activating the one or more TEG components.

Another apparatus is described. The apparatus may include means for receiving temperature data acquired by the one or more sensors, means for determining the temperature difference between the inner ring-shaped housing and the at least one thermally isolated portion of the outer ring-shaped housing based at least in part on the temperature data, and means for activating the one or more TEG components based at least in part on the temperature difference satisfying a threshold temperature difference, wherein generating the electric current is based at least in part on activating the one or more TEG components.

A non-transitory computer-readable medium storing code is described. The code may include instructions executable by one or more processors to receive temperature data acquired by the one or more sensors, determine the temperature difference between the inner ring-shaped housing and the at least one thermally isolated portion of the outer ring-shaped housing based at least in part on the temperature data, and activate the one or more TEG components based at least in part on the temperature difference satisfying a threshold temperature difference, wherein generating the electric current is based at least in part on activating the one or more TEG components.

A method by an apparatus is described. The method may include receiving motion data acquired by the one or more sensors and activating the one or more TEG components based at least in part on the motion data satisfying a threshold motion level, wherein generating the electric current is based at least in part on activating the one or more TEG components.

An apparatus is described. The apparatus may include one or more memories storing processor executable code, and one or more processors coupled with the one or more memories. The one or more processors may individually or collectively operable to execute the code to cause the apparatus to receive motion data acquired by the one or more sensors and activate the one or more TEG components based at least in part on the motion data satisfying a threshold motion level, wherein generating the electric current is based at least in part on activating the one or more TEG components.

Another apparatus is described. The apparatus may include means for receiving motion data acquired by the one or more sensors and means for activating the one or more TEG components based at least in part on the motion data satisfying a threshold motion level, wherein generating the electric current is based at least in part on activating the one or more TEG components.

A non-transitory computer-readable medium storing code is described. The code may include instructions executable by one or more processors to receive motion data acquired by the one or more sensors and activate the one or more TEG components based at least in part on the motion data satisfying a threshold motion level, wherein generating the electric current is based at least in part on activating the one or more TEG components.

A method by an apparatus is described. The method may include monitoring a voltage, a current level, or both, associated with the one or more TEG components and activating the one or more TEG components based at least in part on the voltage, the current level, or both, satisfying one or more thresholds, wherein generating the electric current is based at least in part on activating the one or more TEG components.

An apparatus is described. The apparatus may include one or more memories storing processor executable code, and one or more processors coupled with the one or more memories. The one or more processors may individually or collectively operable to execute the code to cause the apparatus to monitor a voltage, a current level, or both, associated with the one or more TEG components and activate the one or more TEG components based at least in part on the voltage, the current level, or both, satisfying one or more thresholds, wherein generating the electric current is based at least in part on activating the one or more TEG components.

Another apparatus is described. The apparatus may include means for monitoring a voltage, a current level, or both, associated with the one or more TEG components and means for activating the one or more TEG components based at least in part on the voltage, the current level, or both, satisfying one or more thresholds, wherein generating the electric current is based at least in part on activating the one or more TEG components.

A non-transitory computer-readable medium storing code is described. The code may include instructions executable by one or more processors to monitor a voltage, a current level, or both, associated with the one or more TEG components and activate the one or more TEG components based at least in part on the voltage, the current level, or both, satisfying one or more thresholds, wherein generating the electric current is based at least in part on activating the one or more TEG components.

A method by an apparatus is described. The method may include monitoring a voltage, a current level, or both, associated with the one or more TEG components, estimate an additional temperature difference between a skin temperature of the user and a surrounding environment of the user based at least in part on the voltage, the current level, or both, and calibrating one or more skin temperature measurements received from the one or more temperature sensors based at least in part on the additional temperature difference.

An apparatus is described. The apparatus may include one or more memories storing processor executable code, and one or more processors coupled with the one or more memories. The one or more processors may individually or collectively operable to execute the code to cause the apparatus to monitor a voltage, a current level, or both, associated with the one or more TEG components, estimate an additional temperature difference between a skin temperature of the user and a surrounding environment of the user based at least in part on the voltage, the current level, or both, and calibrate one or more skin temperature measurements received from the one or more temperature sensors based at least in part on the additional temperature difference.

Another apparatus is described. The apparatus may include means for monitoring a voltage, a current level, or both, associated with the one or more TEG components, means for estimate an additional temperature difference between a skin temperature of the user and a surrounding environment of the user based at least in part on the voltage, the current level, or both, and means for calibrating one or more skin temperature measurements received from the one or more temperature sensors based at least in part on the additional temperature difference.

A non-transitory computer-readable medium storing code is described. The code may include instructions executable by one or more processors to monitor a voltage, a current level, or both, associated with the one or more TEG components, estimate an additional temperature difference between a skin temperature of the user and a surrounding environment of the user based at least in part on the voltage, the current level, or both, and calibrate one or more skin temperature measurements received from the one or more temperature sensors based at least in part on the additional temperature difference.

An apparatus is described. The apparatus may include an inner ring-shaped housing comprising one or more apertures, wherein the inner ring-shaped housing defines an inner curved surface of the wearable ring device, one or more sensors configured to acquire physiological data from a user through the one or more apertures of the inner ring-shaped housing, an outer ring-shaped housing that at least partially surrounds the inner ring-shaped housing, wherein the outer ring-shaped housing defines an outer curved surface of the wearable ring device, wherein the outer ring-shaped housing comprises at least one thermally isolated portion that extends at least partially around the wearable ring device, wherein the at least one thermally isolated portion is thermally isolated from a remaining portion of the outer ring-shaped housing, and one or more TEG components disposed at least partially between the inner ring-shaped housing and the at least one thermally isolated portion of the outer ring-shaped housing, wherein the one or more TEG components are configured to generate an electric current to power the one or more sensors, recharge a battery of the wearable ring device, or both, based at least in part on a temperature difference between the inner ring-shaped housing and the at least one thermally isolated portion of the outer ring-shaped housing.

In some examples of the apparatus, the one or more sensors, one or more processors of the wearable ring device, or both, may be positioned against the inner ring-shaped housing such that heat generated by the one or more sensors, one or more processors, or both, heats up the inner ring-shaped housing to increase the temperature difference between the inner ring-shaped housing and the at least one thermally isolated portion of the outer ring-shaped housing.

In some examples of the apparatus, the one or more sensors may be positioned within a first radial span of a circumference of the wearable ring device that may be radially opposite of the at least one thermally isolated portion of the outer ring-shaped housing.

In some examples of the apparatus, the one or more sensors and the at least one thermally isolated portion may be positioned around a circumference of the wearable ring device such that, when the one or more sensors may be positioned on a palm-side of a finger of a user, the at least one thermally isolated portion may be positioned on a dorsal-side of the finger, and the remaining portion of the outer ring-shaped housing may be adjacent to one or more additional fingers that may be adjacent to the finger of the user.

Some examples of the apparatus may further include one or more processors communicatively coupled with the one or more sensors and the one or more TEG components, the one or more sensors comprising one or more temperature sensors, the one or more processors configured to, receive temperature data acquired by the one or more temperature sensors, determine the temperature difference between the inner ring-shaped housing and the at least one thermally isolated portion of the outer ring-shaped housing based at least in part on the temperature data, and activate the one or more TEG components based at least in part on the temperature difference satisfying a threshold temperature difference, wherein the one or more TEG components may be configured to generate the electric current based at least in part on activating the one or more TEG components.

Some examples of the apparatus may further include one or more processors communicatively coupled with the one or more sensors and the one or more TEG components, the one or more sensors comprising one or more motion sensors, the one or more processors configured to, receive motion data acquired by the one or more motion sensors, and activate the one or more TEG components based at least in part on the motion data satisfying a threshold motion level, wherein the one or more TEG components may be configured to generate the electric current based at least in part on activating the one or more TEG components.

Some examples of the apparatus may further include one or more processors communicatively coupled with the one or more TEG components, the one or more processors configured to, monitor a voltage, a current level, or both, associated with the one or more TEG components, and activate the one or more TEG components based at least in part on the voltage, the current level, or both, satisfying one or more thresholds, wherein the one or more TEG components may be configured to generate the electric current based at least in part on activating the one or more TEG components.

Some examples of the apparatus may further include one or more processors communicatively coupled with the one or more sensors and the one or more TEG components, the one or more sensors comprising one or more temperature sensors, the one or more processors configured to, monitor a voltage, a current level, or both, associated with the one or more TEG components, estimate an additional temperature difference between a skin temperature of the user and a surrounding environment of the user based at least in part on the voltage, the current level, or both, and calibrate one or more skin temperature measurements received from the one or more temperature sensors based at least in part on the additional temperature difference.

In some examples of the apparatus, the inner ring-shaped housing may be configured to be heated based at least in part on the inner ring-shaped housing contacting a tissue of the user and the at least one thermally isolated portion of the outer ring-shaped housing may be configured to be cooled based at least in part on the at least one thermally isolated portion being exposed to a surrounding environment of the user.

In some examples of the apparatus, the inner ring-shaped housing may be thermally isolated from the at least one thermally isolated portion and the remaining portion of the outer ring-shaped housing.

Some examples of the apparatus may further include one or more grooves within the outer ring-shaped housing, wherein the one or more grooves may be filled with a thermally-insulating material that may be configured to thermally isolate the at least one thermally isolated portion from the remaining portion of the outer ring-shaped housing.

In some examples of the apparatus, the inner ring-shaped housing, the at least one thermally isolated portion of the outer ring-shaped housing, or both, comprise a textured surface that may be configured to increase the temperature difference between the inner ring-shaped housing and the at least one thermally isolated portion of the outer ring-shaped housing by increasing a surface area of the inner ring-shaped housing, the at least one thermally isolated portion, or both.

In some examples of the apparatus, the at least one thermally isolated portion of the outer ring-shaped housing comprises one or more features configured to maintain a liquid during an evaporation process of the liquid and the evaporation process of the liquid may be configured to reduce a temperature of the at least one thermally isolated portion to increase the temperature difference between the inner ring-shaped housing and the at least one thermally isolated portion of the outer ring-shaped housing.

In some examples of the apparatus, the inner ring-shaped housing and the outer ring-shaped housing comprise a same material or different materials.

Some examples of the apparatus may further include one or more reservoirs positioned between the inner ring-shaped housing and the outer ring-shaped housing, wherein the one or more reservoirs may be at least partially filled with a phase-change material, wherein a change of the phase-change material from a first phase to a second phase may be configured to store energy from the electric current, generate an additional electric current to power the one or more sensors or recharge the battery of the wearable ring device, or both.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an inner ring-shaped housing comprising one or more apertures, wherein the inner ring-shaped housing comprises an inner curved surface of the wearable ring device;
one or more sensors configured to acquire physiological data from a user through the one or more apertures of the inner ring-shaped housing;
an outer ring-shaped housing that at least partially surrounds the inner ring-shaped housing, wherein the outer ring-shaped housing comprises defines an outer curved surface of the wearable ring device, wherein the outer ring-shaped housing comprises at least one thermally isolated portion that extends at least partially around the wearable ring device, wherein the at least one thermally isolated portion is thermally isolated from a remaining portion of the outer ring-shaped housing; and
one or more thermo-electric-generator (TEG) components disposed at least partially between the inner ring-shaped housing and the at least one thermally isolated portion of the outer ring-shaped housing, wherein the one or more TEG components are configured to generate an electric current to power the one or more sensors, recharge a battery of the wearable ring device, or both, based at least in part on a temperature difference between the inner ring-shaped housing and the at least one thermally isolated portion of the outer ring-shaped housing.

2. The wearable ring device of claim 1, wherein the one or more sensors, one or more processors of the wearable ring device, or both, are positioned against the inner ring-shaped housing such that heat generated by the one or more sensors, one or more processors, or both, heats up the inner ring-shaped housing to increase the temperature difference between the inner ring-shaped housing and the at least one thermally isolated portion of the outer ring-shaped housing.

3. The wearable ring device of claim 1, wherein the one or more sensors are positioned within a first radial span of the wearable ring device that is radially opposite of the at least one thermally isolated portion of the outer ring-shaped housing.

4. The wearable ring device of claim 1, wherein the one or more sensors and the at least one thermally isolated portion are positioned radially around the wearable ring device such that, when the one or more sensors are positioned on a palm-side of a finger of a user, the at least one thermally isolated portion is positioned on a dorsal-side of the finger, and the remaining portion of the outer ring-shaped housing is adjacent to one or more additional fingers that are adjacent to the finger of the user.

5. The wearable ring device of claim 1, further comprising:
one or more processors communicatively coupled with the one or more sensors and the one or more TEG components, the one or more sensors comprising one or more temperature sensors, the one or more processors configured to:
receive temperature data acquired by the one or more temperature sensors;
determine the temperature difference between the inner ring-shaped housing and the at least one thermally isolated portion of the outer ring-shaped housing based at least in part on the temperature data; and
activate the one or more TEG components based at least in part on the temperature difference satisfying a threshold temperature difference, wherein the one or more TEG components are configured to generate the electric current based at least in part on activating the one or more TEG components.

6. The wearable ring device of claim 1, further comprising:
one or more processors communicatively coupled with the one or more sensors and the one or more TEG components, the one or more sensors comprising one or more motion sensors, the one or more processors configured to:
receive motion data acquired by the one or more motion sensors; and
activate the one or more TEG components based at least in part on the motion data satisfying a threshold motion level, wherein the one or more TEG components are configured to generate the electric current based at least in part on activating the one or more TEG components.

7. The wearable ring device of claim 1, further comprising:
one or more processors communicatively coupled with the one or more TEG components, the one or more processors configured to:
monitor a voltage, a current level, or both, associated with the one or more TEG components; and
activate the one or more TEG components based at least in part on the voltage, the current level, or both, satisfying one or more thresholds, wherein the one or more TEG components are configured to generate the electric current based at least in part on activating the one or more TEG components.

8. The wearable ring device of claim 1, further comprising:
one or more processors communicatively coupled with the one or more sensors and the one or more TEG components, the one or more sensors comprising one or more temperature sensors, the one or more processors configured to:
monitor a voltage, a current level, or both, associated with the one or more TEG components;
estimate an additional temperature difference between a skin temperature of the user and a surrounding environment of the user based at least in part on the voltage, the current level, or both; and
calibrate one or more skin temperature measurements received from the one or more temperature sensors based at least in part on the additional temperature difference.

9. The wearable ring device of claim 1, wherein the inner ring-shaped housing is configured to be heated based at least in part on the inner ring-shaped housing contacting a tissue of the user, and wherein the at least one thermally isolated portion of the outer ring-shaped housing is configured to be cooled based at least in part on the at least one thermally isolated portion being exposed to a surrounding environment of the user.

10. The wearable ring device of claim 1, wherein the at least one thermally isolated portion of the outer ring-shaped housing is configured to be heated based at least in part on the at least one thermally isolated portion being exposed to a surrounding environment of the user, and wherein the inner ring-shaped housing is configured to be cooled relative to the surrounding environment based at least in part on the inner ring-shaped housing contacting a tissue of the user.

11. The wearable ring device of claim 1, wherein the inner ring-shaped housing is thermally isolated from the at least one thermally isolated portion and the remaining portion of the outer ring-shaped housing.

12. The wearable ring device of claim 1, further comprising:
one or more grooves within the outer ring-shaped housing, wherein the one or more grooves are filled with a thermally-insulating material that is configured to thermally isolate the at least one thermally isolated portion from the remaining portion of the outer ring-shaped housing.

13. The wearable ring device of claim 1, wherein the inner ring-shaped housing, the at least one thermally isolated portion of the outer ring-shaped housing, or both, comprise a textured surface that is configured to increase the temperature difference between the inner ring-shaped housing and the at least one thermally isolated portion of the outer ring-shaped housing by increasing a surface area of the inner ring-shaped housing, the at least one thermally isolated portion, or both.

14. The wearable ring device of claim 1, wherein the at least one thermally isolated portion of the outer ring-shaped housing comprises one or more features configured to maintain a liquid during an evaporation process of the liquid, wherein the evaporation process of the liquid is configured to reduce a temperature of the at least one thermally isolated portion to increase the temperature difference between the inner ring-shaped housing and the at least one thermally isolated portion of the outer ring-shaped housing.

15. The wearable ring device of claim 1, wherein the inner ring-shaped housing and the outer ring-shaped housing comprise a same material or different materials.

16. The wearable ring device of claim 1, further comprising:
one or more reservoirs positioned between the inner ring-shaped housing and the outer ring-shaped housing, wherein the one or more reservoirs are at least partially filled with a phase-change material, wherein a change of the phase-change material from a first phase to a second phase is configured to store energy from the electric current, generate an additional electric current to power the one or more sensors or recharge the battery of the wearable ring device, or both.

17. A method for operating a wearable ring device, comprising:
collecting physiological data associated with a user via one or more sensors of the wearable ring device, wherein the wearable ring device comprises an inner ring-shaped housing and an outer ring-shaped housing, wherein the outer ring-shaped housing comprises at least one thermally isolated portion that extends at least partially around the wearable ring device, wherein the at least one thermally isolated portion is thermally isolated from a remaining portion of the outer ring-shaped housing;
generating, using one or more thermo-electric-generator (TEG) components disposed at least partially between the inner ring-shaped housing and the at least one thermally isolated portion of the outer ring-shaped housing, an electric current based at least in part on a temperature difference between the inner ring-shaped housing and the at least one thermally isolated portion of the outer ring-shaped housing; and
recharging a battery of the wearable ring device, powering the one or more sensors, or both, using the electric current.

18. The method of claim 17, further comprising:
activating the one or more TEG components based at least in part on the physiological data collected via the one or more sensors of the wearable ring device, wherein the physiological data comprises least temperature data, motion data, or both, and wherein generating the electric current is based at least in part on activating the one or more TEG components.

19. The method of claim 17, further comprising:
determining that the temperature difference between the inner ring-shaped housing and the at least one thermally isolated portion of the outer ring-shaped housing satisfies a threshold; and
converting a voltage outputted by the one or more TEG components to a usable voltage associated with the electric current based at least in part on the temperature difference satisfying the threshold.

20. The method of claim 17, wherein the physiological data comprises temperature data, the method further comprising:
receiving temperature data acquired by the one or more sensors;
determining the temperature difference between the inner ring-shaped housing and the at least one thermally isolated portion of the outer ring-shaped housing based at least in part on the temperature data; and
activating the one or more TEG components based at least in part on the temperature difference satisfying a threshold temperature difference, wherein generating the electric current is based at least in part on activating the one or more TEG components.
